SEQUENCE LISTING (14 sequences)

SEQ ID NO: 1 - DNA, 52 bp, synthetic construct
  source 1..52: mol_type = other DNA
acgcaaaccgcctgcaggtctagatcttcatcggtgattgattcctttaaag

SEQ ID NO: 2 - DNA, 42 bp, synthetic construct
  source 1..42: mol_type = other DNA
tctcagactcgtctcctctacgaccgcttgccgttaaaagcc

SEQ ID NO: 3 - DNA, 52 bp, synthetic construct
  source 1..52: mol_type = other DNA
ggactagtctagacgtctcatagatcttcatcggtgattgattcctttaaag

SEQ ID NO: 4 - DNA, 42 bp, synthetic construct
  source 1..42: mol_type = other DNA
tctcagactgctcttccctacgaccgcttgccgttaaaagcc

SEQ ID NO: 5 - DNA, 52 bp, synthetic construct
  source 1..52: mol_type = other DNA
gactagtctagagctcttcatagatcttcatcggtgattgattcctttaaag

SEQ ID NO: 6 - DNA, 42 bp, synthetic construct; identical to SEQ ID NO: 2
  source 1..42: mol_type = other DNA

SEQ ID NO: 7 - DNA, 20 bp, synthetic construct
  source 1..20: mol_type = other DNA
gaagtccagctgccagaaac

SEQ ID NO: 8 - DNA, 20 bp, synthetic construct
  source 1..20: mol_type = other DNA
gcaccatcgtcaaccactac

SEQ ID NO: 9 - DNA, 20 bp, synthetic construct
  source 1..20: mol_type = other DNA
caagctagctcacacggttc

SEQ ID NO: 10 - DNA, 20 bp, synthetic construct
  source 1..20: mol_type = other DNA
tgatcttgatgcctccacgt

SEQ ID NO: 11 - DNA, 20 bp, synthetic construct
  source 1..20: mol_type = other DNA
gtgagctgacaatgttgcct

SEQ ID NO: 12 - DNA, 20 bp, synthetic construct
  source 1..20: mol_type = other DNA
gtcatgttgaagagcggcat

SEQ ID NO: 13 - DNA, 20 bp, synthetic construct
  source 1..20: mol_type = other DNA
gttgggtggctcgtcaattt

SEQ ID NO: 14 - DNA, 20 bp, synthetic construct
  source 1..20: mol_type = other DNA
tctttggtatcttgctgcgc

(19) United States
(12) Patent Application Publication
(10) Pub. No.: US 2024/0110196 A1
(43) Pub. Date: Apr. 4, 2024

(54) USE OF MULTIGENE STACKING METHOD IN SYNTHESIS OF NERVONIC ACID IN BRASSICA NAPUS

(71) Applicant: Oil Crops Research Institute, CAAS, Wuhan, Hubei (CN)

(72) Inventors: Fang LIU, Wuhan (CN); Pandi WANG, Wuhan (CN); Gang WU, Wuhan (CN); Xiaojuan XIONG, Wuhan (CN)

(73) Assignee: Oil Crops Research Institute, CAAS, Wuhan, Hubei (CN)

(21) Appl. No.: 18/458,027

(22) Filed: Aug. 29, 2023

(30) Foreign Application Priority Data
Aug. 29, 2022 (CN) 202211039232.9

(51) Int. Cl. C12N 15/82; C12N 9/10

(52) U.S. Cl. CPC C12N 15/8234; C12N 9/1029; C12N 15/8243; C12Y 203/0102; C12Y 203/01051; C12Y 203/01199

(57) ABSTRACT

The present disclosure relates to use of a multigene stacking method in synthesis of nervonic acid in Brassica napus. The present disclosure provides a multigene co-expression plant vector, including an initial backbone of pBWA(V)BII and multiple exogenous gene expression cassettes. The present disclosure further provides two plant vectors applicable to genetic transformation, including a three-gene co-expression plant vector and a five-gene co-expression plant vector. In the present disclosure, after the three-gene co-expression plant vector and the five-gene co-expression plant vector are separately transferred into the Brassica napus, the nervonic acid (NA) with a high content is synthesized in the Brassica napus through multigene co-expression. An NA ratio can be significantly increased combined with a higher seed oil content and a greater field seed yield. The plant vector realizes efficient synthesis of NA in oil crops, obtains NA-rich seed oil, and increases an added value of edible oil.

### CROSS REFERENCE TO RELATED APPLICATION

This patent application claims the benefit and priority of Chinese Patent Application No. 202211039232.9, filed with the China National Intellectual Property Administration on Aug. 29, 2022, the disclosure of which is incorporated by reference herein in its entirety as part of the present application.

### REFERENCE TO ELECTRONIC SEQUENCE LISTING

The application contains a Sequence Listing which has been submitted electronically in .XML format and is hereby incorporated by reference in its entirety. Said .XML copy, created on Aug. 24, 2022, is named “Sequence_Listing.xml” and is 13,272 bytes in size. The sequence listing contained in this .XML file is part of the specification and is hereby incorporated by reference herein in its entirety.

### TECHNICAL FIELD

The present disclosure belongs to the technical field of genetic transformation, and in particular relates to use of a multigene stacking method in synthesis of nervonic acid in Brassica napus.

### BACKGROUND

Nervonic acid (NA; 24:1Δ15, 24:1ω-9; cis-15-tetracosenoic acid) is a very-long-chain monounsaturated fatty acid (VLCMFA, containing 22 to 26 carbon atoms). The NA plays a special role in promoting the repair and regeneration of nerve fibers in damaged brain tissue. NA is combined with sphingosine through an amide bond to form sphingolipids, which mainly exist in the white matter of brain tissue and constitutes the myelin sheath of nerve fibers. The NA plays a vital role in the development and maintenance of brain as well as the biosynthesis and improvement of nerve cells.

Due to the huge market and commercial value of NA, developed countries obtained NA by capturing a large number of sharks in the past few decades. However, international organizations have banned shark fishing. The NA can also be synthesized chemically by using methyl cis-13-docosenoate as a precursor, with a low yield and many by-products. NA is also present in the seed oils of some wild plants. However, the long growth cycle, extremely limited distribution, as well as low and unstable seed yield hinder the extraction of NA from natural plant sources. For example, the industrialization of NA in China mainly focuses on the development of seed oil from Acer truncatum. However, this plant has a low NA content of only about 6%, and can only grow in the north, taking 8 to 10 years to mature. Therefore, it is an effective way to achieve large-scale production in the future by constructing an efficient NA synthesis pathway in oil crops by means of synthetic biology.

Genetic engineering strategies can break through the above bottlenecks and breed new varieties with excellent traits and ideal NA content that are difficult to obtain by traditional breeding methods. The production of microorganisms, microalgae, and plants with a high NA content by genetic engineering provides a suitable method for the nutritional and pharmacological applications of NA, and has attracted widespread attention due to its great potential. The synthesis of NA using microorganisms as a framework is also scalable, but is mainly limited by the capital expenditure of large-scale biological fermentation facilities, and has a cost increasing linearly. The characteristic of synthesizing NA with plants as a framework is that once a mature system is established, a large number of target products can be produced through standard agricultural planting processes, with high initial cost by low later cost. As a result, genetic engineering of plants shows specific advantages. Once a mature system for producing NA is established through genetic engineering in plants, mass production only requires a larger planting area. However, the current technology is still immature on how to build plant biochemical factories. Moreover, even if a plant biochemical factory can be constructed, the expressed NA shows a relatively low yield.

### SUMMARY

A purpose of the present disclosure is to provide use of a multigene stacking method in synthesis of nervonic acid in Brassica napus. The present disclosure is intended to improve a nutritional quality of crops or create plant biochemical factories to produce the NA for nutritional, pharmaceutical, and chemical industries.

The present disclosure provides a multigene co-expression plant vector, including an initial backbone of pBWA(V)BII and multiple exogenous gene expression cassettes; where
an exogenous gene in each of the exogenous gene expression cassettes is activated by a seed-specific expression promoter.

Preferably, the multiple exogenous gene expression cassettes are located in an independent T-DNA region of the plant vector, and the exogenous gene in each of the exogenous gene expression cassettes is a gene related to synthesis and assembly of nervonic acid and grease.

Preferably, the gene related to the synthesis and the assembly of the nervonic acid and the grease includes a 3-ketoacyl-CoA synthase gene, a lysophosphatidic acid acyltransferase gene, and a diacylglycerol acyltransferase gene.

Preferably, the 3-ketoacyl-CoA synthase gene includes BnFAE1 and/or CgKCS; the lysophosphatidic acid acyltransferase gene includes SLC1-1 and/or LdLPAAT; and the diacylglycerol acyltransferase gene includes DGAT1.

The present disclosure further provides a three-gene co-expression plant vector for induced expression of nervonic acid and grease, where the three-gene co-expression plant vector is constructed based on an initial backbone of pBWA(V)BII and three exogenous gene expression cassettes located in a T-DNA region; and
the three exogenous gene expression cassettes include one 3-ketoacyl-CoA synthase gene expression cassette, one lysophosphatidic acid acyltransferase gene expression cassette, and one diacylglycerol acyltransferase gene expression cassette.

Preferably, the 3-ketoacyl-CoA synthase gene expression cassette includes a Napin promoter, a CgKCS gene, and a Napin terminator;
the lysophosphatidic acid acyltransferase gene expression cassette includes a Napin promoter, an SLC1-1 gene, and a Napin terminator; and
the diacylglycerol acyltransferase gene expression cassette includes a Napin promoter, a DGAT1 gene, and a Napin terminator.

The present disclosure further provides a five-gene co-expression plant vector for induced expression of nervonic acid and grease, where the five-gene co-expression plant vector is constructed based on an initial backbone of pBWA(V)BII and five exogenous gene expression cassettes located in a T-DNA region; and
the five exogenous gene expression cassettes include two 3-ketoacyl-CoA synthase gene expression cassettes, two lysophosphatidic acid acyltransferase gene expression cassettes, and one diacylglycerol acyltransferase gene expression cassette.

Preferably, 3-ketoacyl-CoA synthase genes in the two 3-ketoacyl-CoA synthase gene expression cassettes are a CgKCS gene and a BnFAE1 gene, respectively;
lysophosphatidic acid acyltransferase genes in the two lysophosphatidic acid acyltransferase gene expression cassettes are an SLC1-1 gene and a LdLPAAT gene, respectively; and
a diacylglycerol acyltransferase gene in the diacylglycerol acyltransferase gene expression cassette is a DGAT1 gene.

The present disclosure further provides use of the plant vector or the three-gene co-expression plant vector or the five-gene co-expression plant vector in construction of a plant germplasm with high expression of nervonic acid and grease.

Preferably, the plant germplasm includes a Brassica napus germplasm.

Beneficial effects: the present disclosure provides a multigene co-expression plant vector, including an initial backbone of pBWA(V)BII, a Bar gene expression cassette, and multiple exogenous gene expression cassettes. The present disclosure further provides two plant vectors applicable to genetic transformation, including a three-gene co-expression plant vector and a five-gene co-expression plant vector. The three-gene co-expression plant vector includes one 3-ketoacyl-CoA synthase gene expression cassette, one lysophosphatidic acid acyltransferase gene expression cassette, and one diacylglycerol acyltransferase gene expression cassette. The five-gene co-expression plant vector includes two 3-ketoacyl-CoA synthase gene expression cassettes, two lysophosphatidic acid acyltransferase gene expression cassettes, and one diacylglycerol acyltransferase gene expression cassette. In the present disclosure, after the three-gene co-expression plant vector is transferred into the Brassica napus, the nervonic acid (NA) with a high content is synthesized in the Brassica napus through multigene co-expression. An NA ratio can be up to 45.84% with a higher seed oil content (47%) and a greater field seed yield (175 kg/mu). An estimated NA yield is 215 g/kg (47%*45.8%*1000), showing a major breakthrough. This not only surpasses a highest level of NA yield produced by genetic engineering technology in Brassica napus, but also surpasses a highest level of NA yield produced by the best other species through genetic engineering technology. The plant vector realizes efficient synthesis of NA in oil crops, obtains NA-rich seed oil, and increases an added value of edible oil. This technology serves the applications of nutrition and pharmacology, and achieves the transformation in modem agriculture from “production-increasing orientation” to “quality-improving orientation”.

### BRIEF DESCRIPTION OF THE DRAWINGS

To describe the technical solutions in embodiments of the present disclosure or in the prior art more clearly, the accompanying drawings required in the embodiments are briefly described below. Apparently, the accompanying drawings in the following description show merely some embodiments of the present disclosure, and other drawings can be derived from these accompanying drawings by those of ordinary skill in the art without creative efforts.

FIG. 1 shows a vector map of pBWA(V)BII;

FIG. 2 shows a vector map of pBWD(LA)C;

FIG. 3 shows a vector map of pBWD(LB)C;

FIG. 4 shows a plasmid map of Napin-3;

FIG. 5 shows a plasmid map of Napin-5;

FIGS. 6A-E show positive identification results of a T0 transgenic line of Napin-3; where FIG. 6A, FIG. 6B, FIG. 6C, and FIG. 6D are results of PCR detection of Bar, CgKCS, SLC1-1 and DGAT1 gene, which are M (Marker, DL2000 DNA ladder), Napin-3 T0 transformation individual plants No. 1 to No. 23, and a negative control (Brassica juncea sp. yellow seed) from left to right; and FIG. 6E is test strip detection results, which are the Napin-3 T0 transformation individual plants No. 1 to No. 23 and the negative control (Brassica juncea sp. yellow seed, represented by N) from left to right;

FIGS. 7A-B show positive identification results of a T0 transgenic line of Napin-5; where FIG. 7A is PCR detection results of Bar gene, and FIG. 7B is test strip detection results; and in FIG. 7A to FIG. 7B, there are M (Marker, DL2000 DNA ladder), Napin-5 T0 transformation individual plants No. 1 to No. 23, and a negative control (Brassica juncea sp. yellow seed, represented by N) from left to right;

FIGS. 8A-D show a result of fatty acid composition analyzed by gas chromatography (GC); where FIG. 8A is GC analysis for 37 kinds of fatty acid methyl ester mixed standards+nervonic acid standard sample; FIG. 8B is GC analysis for seeds of Brassica juncea sp. yellow seed (control); FIG. 8C is GC analysis for seeds of transgenic Napin-3 Brassica juncea sp. yellow seed; and FIG. 8D is a proportion of nervonic acid in seeds (T1 generation) of 34 T0 generation positive individual plants;

FIG. 9A shows a change of fatty acid composition of seed oil (T1 generation) of a Napin-3 transgenic T0 generation positive individual plant compared to that of a wild type; FIG. 9B shows a change of fatty acid composition of seed oil (T2 generation) of a Napin-3 transgenic T1 generation positive individual plant compared to that of a wild type;

FIGS. 10A-D show a proportion of nervonic acid in seeds (T1 generation) of 13 T0 generation positive individual plants; where FIG. 10A is GC analysis for 37 kinds of fatty acid methyl ester mixed standards+nervonic acid standard sample; FIG. 10B is GC analysis for seeds of Brassica juncea sp. yellow seed (control); FIG. 10C is GC analysis for seeds of transgenic Napin-5 Brassica juncea sp. yellow seed; and FIG. 10D is a proportion of nervonic acid in seeds (T1 generation) of 13 T0 generation positive individual plants; and

FIG. 11A shows a change of fatty acid composition of seed oil (T1 generation) of a Napin-5 transgenic T0 generation positive individual plant compared to that of a wild type; FIG. 11B shows a change of fatty acid composition of seed oil (T2 generation) of a Napin-5 transgenic T1 generation positive individual plant compared to that of a wild type.

### DETAILED DESCRIPTION OF THE EMBODIMENTS

The present disclosure provides a multigene co-expression plant vector, including an initial backbone of pBWA(V)BII and multiple exogenous gene expression cassettes; where
an exogenous gene in each of the exogenous gene expression cassettes is activated by a seed-specific expression promoter.

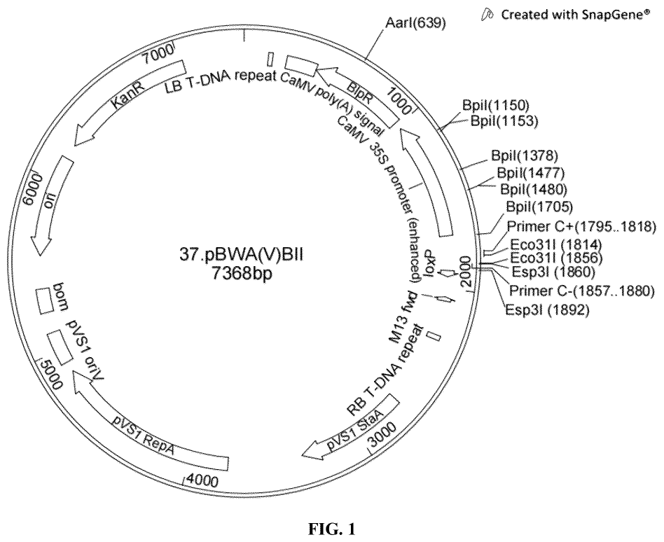

In the present disclosure, the plant vector is constructed based on the initial backbone of pBWA(V)BII (FIG. 1) and an additional synthetic fragment inserted into a multiple cloning site, where the additional synthetic fragment includes a gene expression cassette. The plant vector constructed by the above construction method includes preferably an independent T-DNA region. The T-DNA region includes the Bar gene expression cassette (with the initial backbone of pBWA(V)BII vector) and several exogenous gene expression cassettes, and exogenous genes in the exogenous gene expression cassettes each are genes related to the synthesis and assembly of nervonic acid and grease. The Bar gene expression cassette includes preferably a CaMV 35S promoter, a Bar gene, and a CAMV PolyA terminator that are constitutively expressed. This expression cassette has a full length of 1,455 bp, and is a part of pBWA(V)BII itself. The Bar gene (JQ293091.1) from Streptomyces hygroscopicus encodes phosphinothricin acetyltransferase, which confers resistance to a herbicide glufosinate-ammonium in plants. This gene is a selectable marker gene.

In the present disclosure, the gene related to the synthesis and the assembly of the nervonic acid and the grease includes preferably a 3-ketoacyl-CoA synthase gene, a lysophosphatidic acid acyltransferase gene, and a diacylglycerol acyltransferase gene; where the 3-ketoacyl-CoA synthase gene includes preferably BnFAE1 and/or CgKCS; the lysophosphatidic acid acyltransferase gene includes preferably SLC1-1 and/or LdLPAAT; and the diacylglycerol acyltransferase gene includes preferably DGAT1. The plant vector includes gene expression cassettes that are constructed based on the genes related to the synthesis and the assembly of the nervonic acid and the grease, specifically including two 3-ketoacyl-CoA synthase gene expression cassettes, two lysophosphatidic acid acyltransferase gene expression cassettes, and one diacylglycerol acyltransferase gene expression cassette.

In the present disclosure, each of the gene expression cassettes includes preferably a seed-specific expression promoter, a corresponding gene, and a terminator. For example, the 3-ketoacyl-CoA synthase gene expression cassette includes a CgKCS gene expression cassette and a BnFAE1 gene expression cassette. The CgKCS gene expression cassette includes a Napin promoter, a CgKCS gene, and a Napin terminator for seed-specific expression, with a full length of 2,878 bp. The CgKCS gene (EU871788.1) from Cardamine graeca encodes 3-ketoacyl-CoA synthase, which can improve an efficiency of synthesizing substrates into NA. The BnFAE1 gene expression cassette includes a Napin promoter, a BnFAE1 gene, and a Napin terminator for seed-specific expression, with a full length of 2,878 bp. The BnFAE1 gene (AF274750.1) from Brassica napus L. encodes 3-ketoacyl-CoA synthase, which can increase the synthesis of long-chain fatty acids and increase the number of NA substrates.

In the present disclosure, the lysophosphatidic acid acyltransferase gene expression cassette includes preferably a SLC1-1 gene expression cassette and an LdLPAAT gene expression cassette. The SLC1-1 gene expression cassette includes preferably a Napin promoter, an SLC1-1 gene, and a Napin terminator for seed-specific expression, with a full length of 2,269 bp. The SLC1-1 gene (JQ844755.1) from yeast (Saccharomyces cerevisiae) encodes lysophosphatidic acid acyltransferase, which can improve an efficiency of NA integration into an sn-2 position of triglyceride and increase an oil content. The LdLPAAT gene expression cassette includes preferably a Napin promoter, an LdLPAAT gene, and a Napin terminator for seed-specific expression, with a full length of 2,203 bp. The LdLPAAT gene (DQ402047.1) from Limnanthes douglasii encodes lysophosphatidic acid acyltransferase, which can improve an efficiency of NA integration into an sn-2 position of triglyceride and increase an oil content.

In the present disclosure, the diacylglycerol acyltransferase gene expression cassette includes preferably a DGAT1 gene expression cassette. The DGAT1 gene expression cassette includes preferably a Napin promoter, a DGAT1 gene, and a Napin terminator for seed-specific expression, with a full length of 2,920 bp. The DGAT1 gene (BT008883.1) from Arabidopsis thaliana encodes diacylglycerol acyltransferase, which can improve an efficiency of NA integration into an sn-3 position of triglyceride and increase an oil content.

In the present disclosure, each of the exogenous gene expression cassettes is preferably inserted into a multiple cloning site (MCS) of the vector backbone.

The present disclosure further provides a three-gene co-expression plant vector for induced expression of nervonic acid and grease, where the three-gene co-expression plant vector is constructed based on an initial backbone of pBWA(V)BII and three exogenous gene expression cassettes located in a T-DNA region; and

The three exogenous gene expression cassettes include one diacylglycerol acyltransferase gene expression cassette.

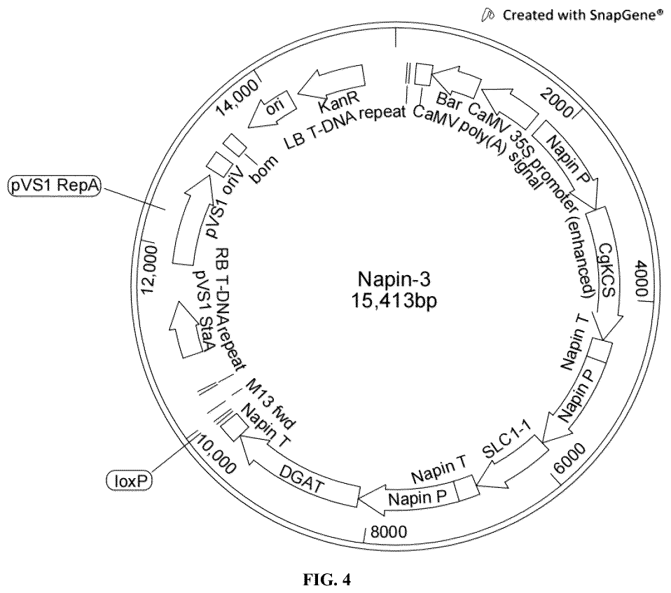

In the present disclosure, the three-gene co-expression plant vector (Napin-3) has a plasmid map preferably shown in FIG. 4; the 3-ketoacyl-CoA synthase gene expression cassette involved is preferably a CgKCS gene expression cassette; and the CgKCS gene expression cassette is preferably the same as that described above, and will not be repeated here. In the three-gene co-expression plant vector, the lysophosphatidic acid acyltransferase gene expression cassette involved is preferably an SLC1-1 gene expression cassette; and the SLC1-1 gene expression cassette is preferably the same as that described above, and will not be repeated here. In the three-gene co-expression plant vector, the diacylglycerol acyltransferase gene expression cassette involved is preferably a DGAT1 gene expression cassette; and the DGAT1 gene expression cassette is preferably the same as that described above, and will not be repeated here.

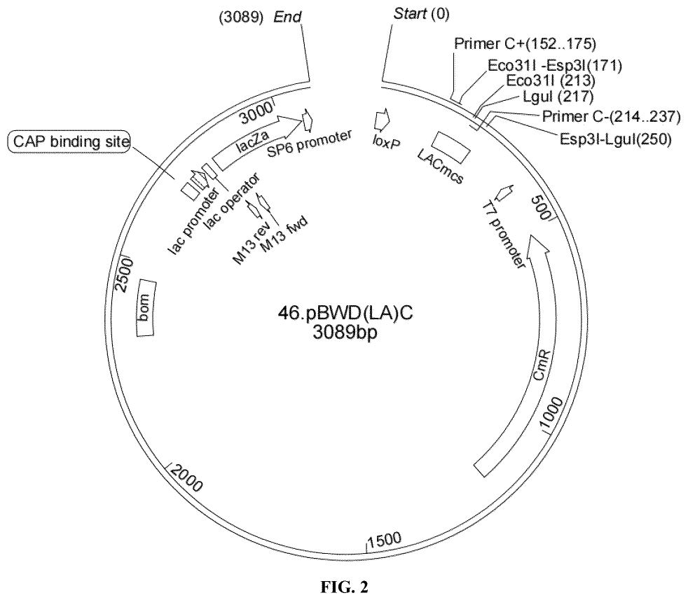

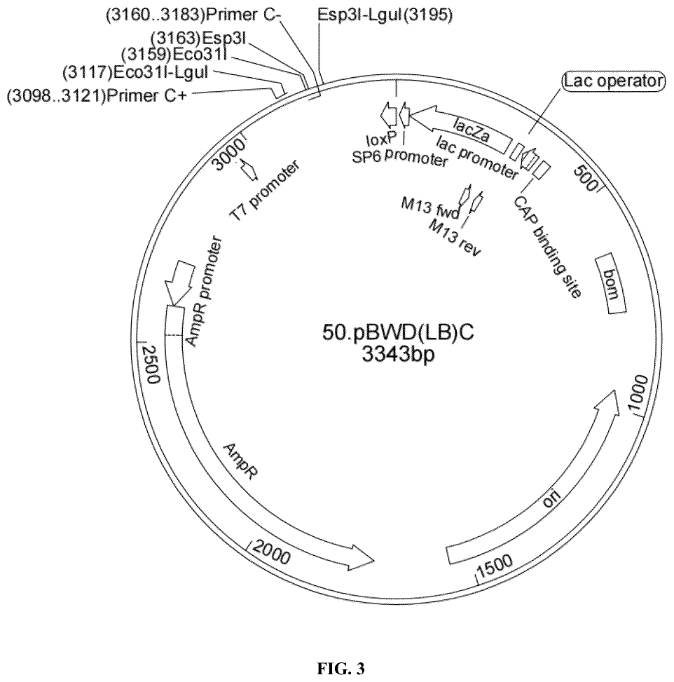

The present disclosure further provides a construction method of the Napin-3. The vector construction is entrusted to Wuhan Biorun Biotechnology Co., Ltd. The involved vectors pBWA(V)BII, pBWD(LA)C (FIG. 2), and pBWD(LB)C (FIG. 3) are also provided by Wuhan Biorun Biotechnology Co., Ltd. The construction method includes preferably the following steps:
(1) The following primers were synthesized:

3300-1-L
(SEQ ID NO. 1):
acgcaaaccgcctgcaggtctagaTCTTCATCGGTGATTGATTCCTTTAA
AG;
3300-1-R
(SEQ ID NO. 2):
tctcagactcgtctcctctacgacCGCTTGCCGTTAAAAGCC;
LAC-L
(SEQ ID NO. 3):
ggactagtctagacgtctcatagaTCTTCATCGGTGATTGATTCCTTTAA
AG;
LAC-R
(SEQ ID NO. 4):
tctcagactgctcttccctacgacCGCTTGCCGTTAAAAGCC;
LBC-L
(SEQ ID NO. 5):
gactagtctagagctcttcatagaTCTTCATCGGTGATTGATTCCTTTAA
AG;
LBC-R
(SEQ ID NO.6):
tctcagactcgtctcctctacgacCGCTTGCCGTTAAAAGCC.

In the present disclosure, when the above primers are designed: the above left primer (-L) including a homology arm of the pBWA(V)BII vector and a Napin promoter in the gene expression cassette are used as the beginning; the above right primer (-R) including the homology arm of the pBWA(V)BII vector and a Napin terminator in the gene expression cassette as the end.
(2) The above three pairs of primers are amplified by PCR to obtain the CgKCS gene expression cassette, the SLC1-1 gene expression cassette, and the DGAT1 gene expression cassette. The above amplification products are gelled to recover DNA, and then recombined with pBWA(V)BII, pBWD(LA)C, and pBWD(LB)C vectors, respectively, after correct detection. The constructed vectors are named pBWA(V)BII-CgKCS, pBWD(LA)C-SLC1-1, and pBWD(LB)C-DGAT1. At this point, the three exogenous gene expression cassettes have been ligated to the three vectors, and then only exogenous gene expression cassettes on latter two intermediate vectors need to be transferred to a final vector pBWA(V)BII.
(3) The pBWA(V)BII-CgKCS includes a CgKCS expression cassette (an end of the expression cassette has an Esp3I restriction site). The pBWD(LA)C-SLC1-1 is digested with Esp3I to obtain an SLC1-1 expression cassette (an end of this expression cassette has a LyuI restriction site), which is ligated to pBWA(V)BII-CgKCS digested with the Esp3I to form pBWA(V)BII-CgKCS-SLC1-1. The pBWD(LB)C-DGAT1 is digested with LyuI to obtain a DGAT1 expression cassette, which is ligated to the pBWA(V)BII-CgKCS-SLC1-1 digested with the LyuI to form pBWA(V)BII-CgKCS-SLC1-1-DGAT1, named as Napin-3.

The present disclosure further provides a five-gene co-expression plant vector for induced expression of nervonic acid and grease, where the five-gene co-expression plant vector is constructed based on an initial backbone of pBWA(V)BII and five exogenous gene expression cassettes located in a T-DNA region; and
the five exogenous gene expression cassettes include two 3-ketoacyl-CoA synthase gene expression cassettes, two lysophosphatidic acid acyltransferase gene expression cassettes, and one diacylglycerol acyltransferase gene expression cassette.

In the present disclosure, the five-gene co-expression plant vector (Napin-5) preferably has a plasmid map shown in FIG. 5. The 3-ketoacyl-CoA synthase gene expression cassette involved preferably includes a CgKCS gene expression cassette and a BnFAE1 gene expression cassette. The CgKCS gene expression cassette and the BnFAE1 gene expression cassette are preferably the same as those described above, and will not be repeated here. In the five-gene co-expression plant vector, the lysophosphatidic acid acyltransferase gene expression cassette involved includes preferably an SLC1-1 gene expression cassette and an LdLPAAT gene expression cassette. The SLC1-1 gene expression cassette and the LdLPAAT gene expression cassette are preferably the same as those described above, and will not be repeated here. In the five-gene co-expression plant vector, the diacylglycerol acyltransferase gene expression cassette involved is preferably a DGAT1 gene expression cassette; and the DGAT1 gene expression cassette is preferably the same as that described above, and will not be repeated here.

In the present disclosure, a construction method of the Napin-5 includes preferably:
(1) Amplification is conducted with a primer pair shown in SEQ ID NO: 1 and SEQ ID NO: 2 to obtain the CgKCS gene expression cassette; amplification is conducted with a primer pair shown in SEQ ID NO: 3 and SEQ ID NO: 4 to obtain the two gene expression cassettes SLC1-1 and LdLPAAT; and amplification is conducted with a primer pair shown in SEQ ID NO: 5 and SEQ ID NO: 6 to obtain the two gene expression cassettes BnFAE1 and DGAT1.
(2) The electrophoresis fragments of the three amplified products are cut, and recovered by sol. The recovered DNA is dissolved with ultrapure water. After correct detection, the CgKCS gene expression cassette is recombined with pBWA(V)BII, the SLC1-1 and LdLPAAT gene expression cassettes are separately recombined with the pBWD(LA)C vector, and the BnFAE1 and DGAT1 gene expression cassettes are separately recombined with the pBWD(LB)C vector. The constructed vectors are named pBWA(V)BII-CgKCS, pBWD(LA)C-SLC1-1, pBWD(LA)C-LdLPAAT, pBWD(LB)C-BnFAE1, and pBWD(LB)C-DGAT1.
(3) The pBWA(V)BII-CgKCS includes a CgKCS expression cassette (an end of the expression cassette has an Esp3I restriction site). The pBWD(LA)C-SLC1-1 is digested with the Esp3I to obtain an SLC1-1 expression cassette (an end of the expression cassette has a LyuI restriction site), which is ligated to the pBWA(V)BII-CgKCS digested with Esp3I to form pBWA(V)BII-CgKCS-SLC1-1. The pBWD(LB)C-BnFAE1 is digested with the LyuI to obtain a BnFAE1 expression cassette (an end of the expression cassette has an Esp3I restriction site), which is ligated to the pBWA(V)BII-CgKCS-SLC1-1 digested with LyuI to form pBWA(V)BII-CgKCS-SLC1-1-BnFAE1. The pBWD(LA)C-LdLPAAT is digested with the Esp3I to obtain an LdLPAAT expression cassette (an end of the expression cassette has a LyuI restriction site), which is ligated to the pBWA(V)BII-CgKCS-SLC1-1-BnFAE1 digested with Esp3I to form pBWA(V)BII-CgKCS-SLC1-1-BnFAE1-LdLPAAT. The pBWD(LB)C-DGAT1 is digested with the LyuI to obtain a DGAT1 expression cassette, which is ligated to the pBWA(V)BII-CgKCS-SLC1-1-BnFAE1-LdLPAAT digested with LyuI to form pBWA(V)BIII-CgKCS-SLC1-1-BnFAE1-LdLPAAT-DGAT1, named as Napin-5.

The present disclosure further provides use of the plant vector or the three-gene co-expression plant vector or the five-gene co-expression plant vector in construction of a plant germplasm with high expression of nervonic acid and grease.

In the present disclosure, the three-gene co-expression plant vector or the five-gene co-expression plant vector can be transformed into a recipient material by genetic transformation. The recipient material includes preferably a Brassica napus germplasm, more preferably Brassica juncea sp. yellow seed with a moderate EA content (erucic acid, 38%) and NA (2% to 3%). There is no special limitation on a transformation method, which preferably includes an Agrobacterium-mediated method, and more preferably refers to a method of Liu et al. to conduct genetic transformation of the plant expression vector in Brassica napus (Liu F, Xiong X J, Wu L, Fu D H, Hayward A, Zeng X H, Cao Y L, Wu Y H, Li Y J, & Wu G (2014) BraLTP1, a lipid transfer protein gene involved in epicuticular wax deposition, cell proliferation and flower development in Brassica napus. PLOS ONE (IF 3.534) 9(10): e110272).

In order to further illustrate the present disclosure, the use of a multigene stacking method in synthesis of nervonic acid in Brassica napus provided by the present disclosure are described in detail below in connection with accompanying drawings and examples, but these examples should not be understood as limiting the claimed scope of the present disclosure.

### Example 1

The genetic transformation of a plant expression vector in Brassica napus was conducted referring to a method of Liu et al. (Liu F, Xiong X J, Wu L, Fu D H, Hayward A, Zeng X H, Cao Y L, Wu Y H, Li Y J, & Wu G (2014) BraLTP1, a lipid transfer protein gene involved in epicuticular wax deposition, cell proliferation and flower development in Brassica napus. PLOS ONE (IF 3.534) 9(10): e110272). The method specifically included: Napin-3 (FIG. 4) and Napin-5 (FIG. 5) plasmids were introduced into Agrobacterium tumefaciens GV3101 by electroporation, cultured on an LB agar plate at 37° C., and positive clones were screened to allow PCR verification, where the plate had gentamicin 50 mg/L, rifampicin 50 mg/L, and kanamycin 50 mg/L. Brassica napus was transformed with a single positive Agrobacterium colony, where Brassica juncea sp. yellow seed (short for Yellow Seed) was used as a receptor. Seeds of the Yellow Seed were soaked in 75% ethanol for 1 min, soaked in 1.5% mercuric chloride for 10 min to 15 min, and germinated in the dark for 5 d to 6 d. Etiolated hypocotyls were cut into 7 mm segments, and mixed with 50 mL of Agrobacterium (an OD value of the Agrobacterium was about 0.3) in a liquid DM medium (MS+30 g/L sucrose+100 μM acetosyringone, pH=5.8) and then soaked for 0.5 h. Surface-dried hypocotyls were transferred to a co-culture medium (MS+30 g/L sucrose+18 g/L mannitol+1 mg/L 2,4-D+0.3 mg/L kinetin+100 μM acetosyringone+8.5 g agarose, pH=5.8) for 2 d, and then in a selective medium (MS+30 g/L sucrose+18 g/L mannitol+1 mg/L 2,4-D+0.3 mg/L kinetin+20 mg/L AgNO₃+8.5 g/L agarose+20 mg/L glufosinate-ammonium+250 mg/L carbenicillin disodium, pH=5.8) for proliferation. After 3 weeks, a callus of the hypocotyl was transferred to a regeneration medium (MS+10 g/L glucose+0.25 g/L xylose+0.6 g/L 2-(N-morpholino)ethanesulfonic acid hydrate+2 mg/L zeatin+0.1 mg/L indole-3-acetic acid+8.5 g/L agarose+20 mg/L glufosinate-ammonium+250 mg/L carbenicillin disodium, pH=5.8) and cultured for 2 weeks. The hypocotyls were transferred to a new regeneration medium every 2 weeks, for a total of 3 to 4 regeneration cycles. After emergence, seedlings were transferred to a rooting medium (MS+10 g/L sucrose+10 g/L agar, pH=5.8) for rooting (about 3 weeks). The transformed rooted plants were transplanted into pots for growth.

Transgenic plants with tissue culture resistance were obtained through genetic transformation.

### Molecular Biological Identification of the Transgenic Plants

### 1. Molecular Biological Identification of Napin-3 Transgenic Plants

For candidate plants obtained by transformation, positive plants therein were identified by PCR and test strips.

Positive PCR identification was conducted on the T0 generation transformation lines with tissue culture resistance: a leaf DNA of the transgenic plants was extracted, and PCR detection was conducted using the DNA of wild-type Brassica juncea sp. yellow seed plants as a negative control with a Bar primer, SLC1-1 primer, and a DGAT1 primer.

PCR amplification was conducted with primer pairs SEQ ID NO: 7: 5′-GAAGTCCAGCTGCCAGAAAC-3′ and SEQ ID NO: 8: 5′-GCACCATCGTCAACCACTAC-3′ and the leaf DNA. If a 440 bp fragment was obtained, it proved that the Bar gene existed in the transgenic plant.

PCR amplification was conducted with primer pairs SEQ ID NO: 9: 5′-CAAGCTAGCTCACACGGTTC-3′ and SEQ ID NO: 10: 5′-TGATCTTGATGCCTCCACGT-3′ and the leaf DNA. If a 373 bp fragment was obtained, it proved that the CgKCS gene existed in the transgenic plant.

PCR amplification was conducted with primer pairs SEQ ID NO: 11: 5′-GTGAGCTGACAATGTTGCCT-3′ and SEQ ID NO: 12: 5′-GTCATGTTGAAGAGCGGCAT-3′ and the leaf DNA. If a 320 bp fragment was obtained, it proved that the SLC1-1 gene existed in the transgenic plant.

PCR amplification was conducted with primer pairs SEQ ID NO: 13: 5′-GTTGGGTGGCTCGTCAATTT-3′ and SEQ ID NO: 14: 5′-TCTTTGGTATCTTGCTGCGC-3′ and the leaf DNA. If a 372 bp fragment was obtained, it proved that the DGAT1 gene existed in the transgenic plant.

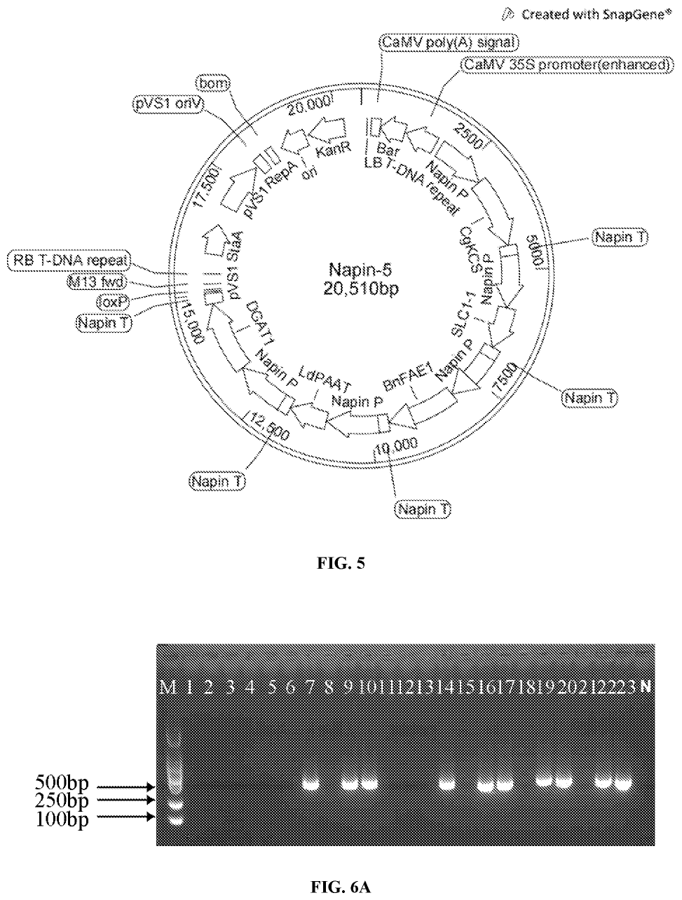

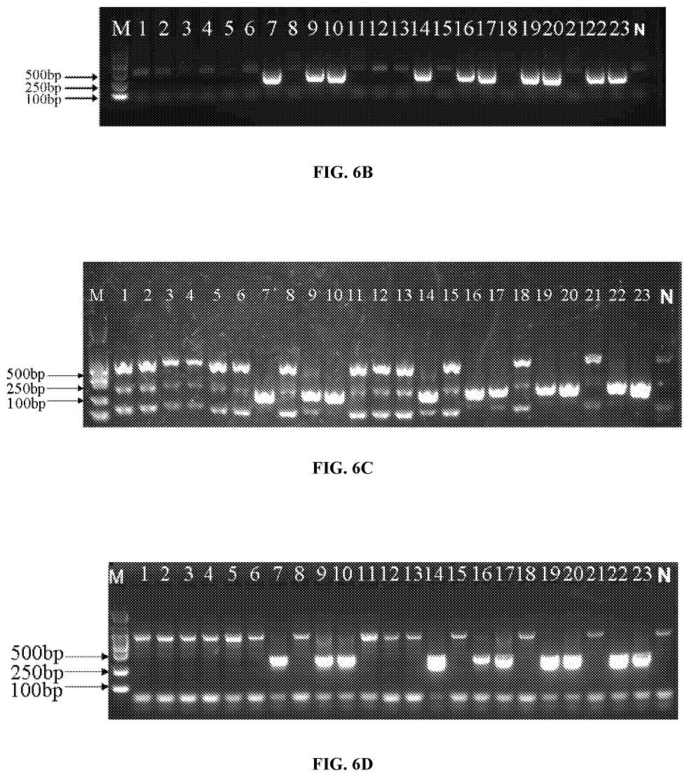

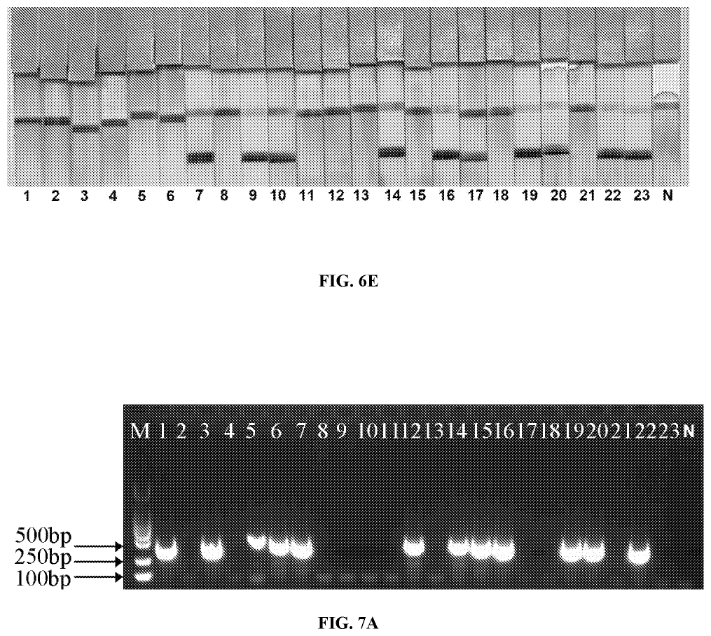

The PCR results were shown in FIGS. 6A-6C, and a total of 10 lines were identified as positive. The 7th, 9th, 10th, 14th, 16th, 17th, 19th, 20th, 22nd, and 23rd Napin-3 transformed lines of the T0 generation had the target band of the Bar gene and CgKCS gene and were positive plants (FIG. 6A-6B). All the Napin-3 transformed lines of the T0 generation and the control (Brassica juncea sp. yellow seed) had the target band of the SLC1-1 gene. However, the target bands in the 7th, 9th, 10th, 14th, 16th, 17th, 19th, 20th, 22nd, and 23rd lines were obviously brighter, which were consistent with the results of the positive lines of the Bar gene. Although the overexpressed SLC1-1 gene came from yeast, rapeseed itself also had a homologous gene, such that there were multiple PCR bands (FIG. 6C). The 7th, 9th, 10th, 14th, 16th, 17th, 19th, 20th, 22nd, and 23rd Napin-3 transformed lines of the T0 generation had the target band of DGAT1, and were positive plants. Since rapeseed itself also had the DGAT1 gene, there were also bands in other regions, but these bands were larger than the overexpressed gene, and could clearly distinguish the transgenic lines (FIG. 6D).

Meanwhile, with the test strip and detection kit developed by the Oil Crops Research Institute of the Chinese Academy of Agricultural Sciences (Wuhan), according to the manufacturer's instructions, the leaves of T0 generation plants of transgenic Brassica napus and non-transgenic Brassica juncea sp. yellow seed (control) were identified by test strips to detect whether the Bar gene expressed protein. The results were shown in FIG. 6E: the 7th, 9th, 10th, 14th, 16th, 17th, 19th, 20th, 22nd, and 23rd lines of the Napin-3 transformed lines of the T0 generation showed two bands on the Bar test strip, and were positive, indicating that the Bar expressed proteins. However, the Brassica juncea sp. yellow seed (control) had only one band, and was negative, indicating that there was no expression of Bar protein.

### 2. Molecular Biological Identification of Napin-5 Transgenic Plants

For candidate plants obtained by transformation, positive plants therein were identified by PCR and test strips.

Leaf DNA extracted from transgenic plants and plant DNA extracted from wild-type Brassica juncea sp. yellow seed as a negative control were subjected to molecular detection. A gene sequence containing five expression cassettes was artificially synthesized, and all target genes and Bar expression cassettes were in one T-DNA region. Therefore, PCR detection was conducted using Bar primers (SEQ ID NO: 7 and SEQ ID NO: 8). If the amplified product was a 440 bp fragment, it proved that the Bar gene existed in the transgenic plants.

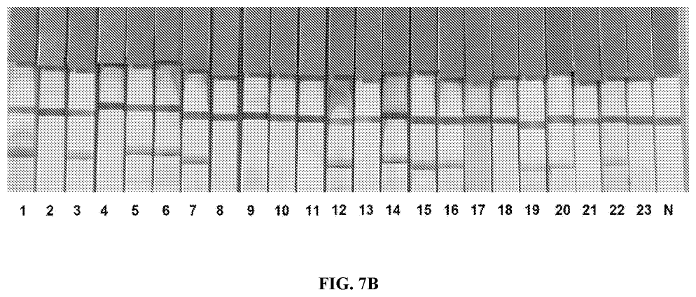

The results were shown in FIGS. 7A-B, a total of 12 lines were identified as positive. The 1st, 3rd, 5th, 6th, 7th, 12th, 14th, 15th, 16th, 19th, 20th, and 22nd Napin-5 transformed lines of the T0 generation had the target band of the Bar gene and were positive plants (FIG. 7A). In the 1st, 3rd, 5th, 6th, 7th, 12th, 14th, 15th, 16th, 19th, 20th, and 22nd lines of the Napin-5 transformed lines of the T0 generation, the Bar test strips showed two bands, which were positive, indicating that the Bar had expressed protein. However, the Brassica juncea sp. yellow seed (control) had only one band, and was negative, indicating that there was no expression of Bar protein (FIG. 7B).

### Example 2 Determination of Fatty Acids in Transgenic Plants

The differences in the components and contents of fatty acids in the seeds of transgenic and wild-type plants were determined by GC. From each material, 5 to 10 rapeseeds were put into a 115 mL centrifuge tube and ground into powder, and 0.6 mL of petroleum ether was added as a solvent to allow ultrasonic extraction for 20 min. After the ultrasonic extraction, 0.25 mL of 04N potassium hydroxide-methanol solution was added, mixed well, and subjected to methyl esterification under ultrasonic for 20 min. After cooling to room temperature, 0.5 mL of distilled water was added, a resulting mixture was shaken vigorously and centrifuged, a supernatant was pipetted into a GC sample bottle, and the sample bottle was put into an autosampler in order for later use.

Quantitative determination of fatty acid methyl esters was completed using GC. A GC instrument was equipped with a hydrogen-flame ionization detector and a DB-23 capillary column (30 m; 0.320 mm; 0.25 μm) from Agilent Technologies, with nitrogen as a carrier at a flow rate of 20 ml/min. A temperature was held at 180° C. for 2 min, then gradually increased to 220° C. at 5° C./min, and finally held at 220° C. for 5 min. One sample was treated from 15 min.

### 1. Determination of Fatty Acids in Napin-3 Transgenic Plants

Various fatty acids in the seeds (T1 generation) of 34 T0 generation positive individuals and in the seeds of Brassica juncea sp. yellow seed individual plants were determined by GC. The phenotypic traits of transgenic crops and recipient Brassica juncea sp. yellow seed were basically indistinguishable. The transferred genes were mainly to improve a nutritional quality of Brassica napus, and the nervonic acid content of transgenic lines was increased by overexpressing the CgKCS, SLC1-1, and DGAT1 genes.

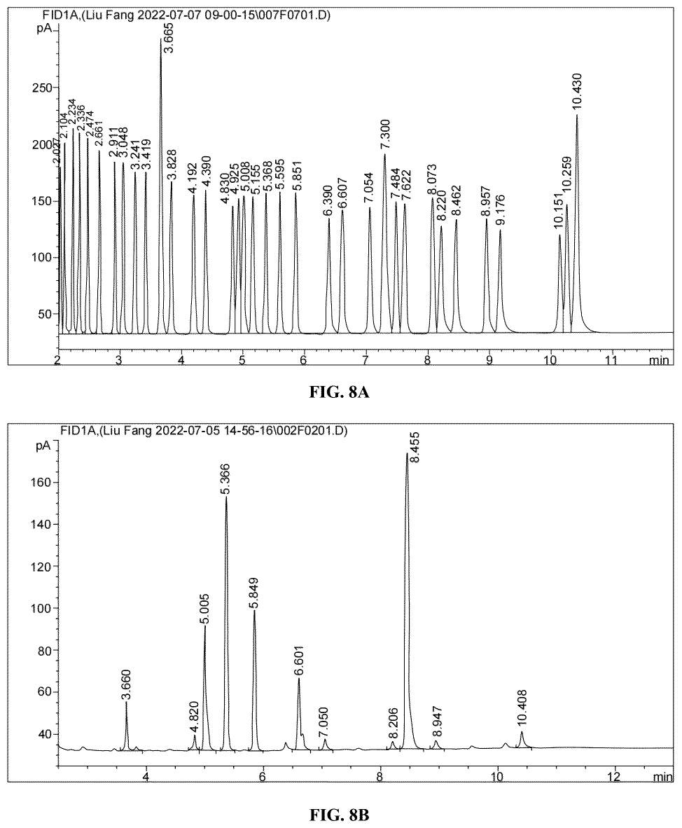

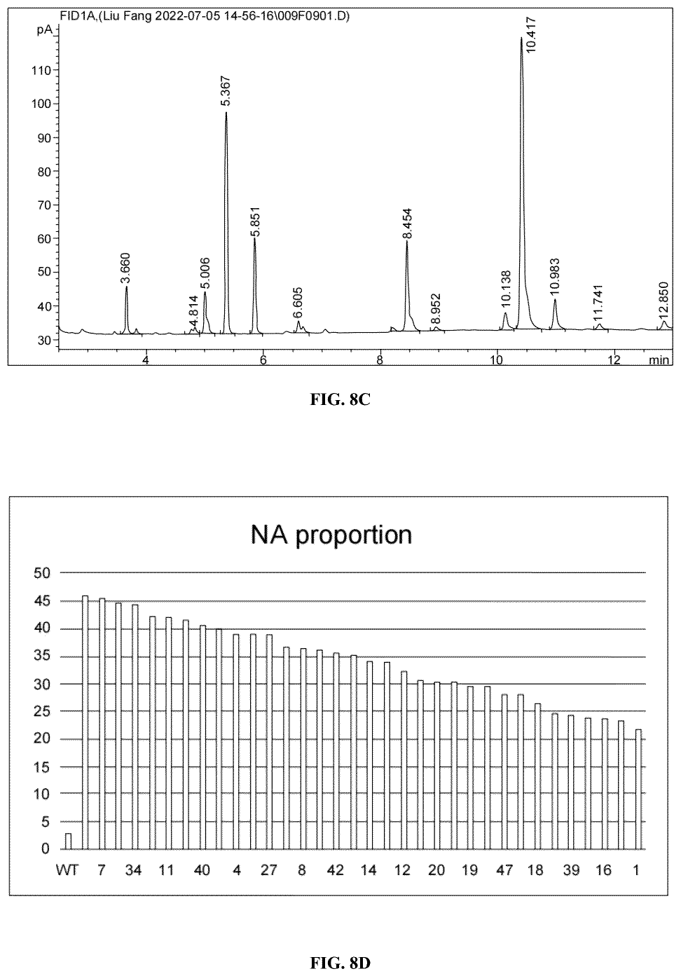

The inventors added an equal volume of 1 mg/ml nervonic acid methyl ester standard sample to the 25 mg/ml of 37 fatty acid methyl ester mixed standards for GC analysis. The additionally increased peak area indicated a peak position of nervonic acid at 10.430 min (FIG. 8A). The fatty acid composition of the control Brassica juncea sp. yellow seed was shown in FIG. 8B, where a peak position of nervonic acid was at 10.408 min. The fatty acid composition of the Napin-3-transgenic Brassica juncea sp. yellow seed control was shown in FIG. 8C, where a peak position of nervonic acid was at 10.417 min. GC results showed that the composition of nervonic acid and other fatty acids in the transgenic lines did change significantly (FIGS. 8A-8C). FIG. 8D showed that a series of T0 generation materials with high NA ratio were obtained: the seed oil in wild-type material had an NA ratio of 2.71%. Among the T0 generation genetically engineered Brassica napus, seed oil in 8 lines had a NA ratio of higher than 40%, 9 lines had a NA ratio of 35% to 40%, and 6 lines had a NA ratio of 30% to 35%. In these lines, NA accounted for 21.57% to 45.84% in fatty acids, and could reach 45.84% at the maximum level. The proportion of NA in fatty acids in T1 generation genetically engineered Brassica napus lines was up to 48.94%.

In the present disclosure, the transgenic receptors (wild-type and control) were Brassica juncea containing moderate erucic acid content and NA, and their oil content was 42% in Hubei and 47% in Northwest China. Yield: this species had a yield of 240 jin/mu in Zhejiang and 350 jin/mu in Gansu, and was more suitable for planting in the northwest. This species was extremely early-maturing, and could reach a yield of 380 jin/mu in high-yield plots in Gansu. Calculated according to the same ratio of T0 generation, an estimated NA yield in seed oil was (193-215) g/kg (45.84%*42 to 47%*1000); calculated according to the same ratio of T1 generation, an estimated NA yield in seed oil was (206-230) g/kg (48.94%*42 to 47%*1000), which was much higher than the highest level reported in plants so far, and might even achieve high production of NA considering the yield factor.

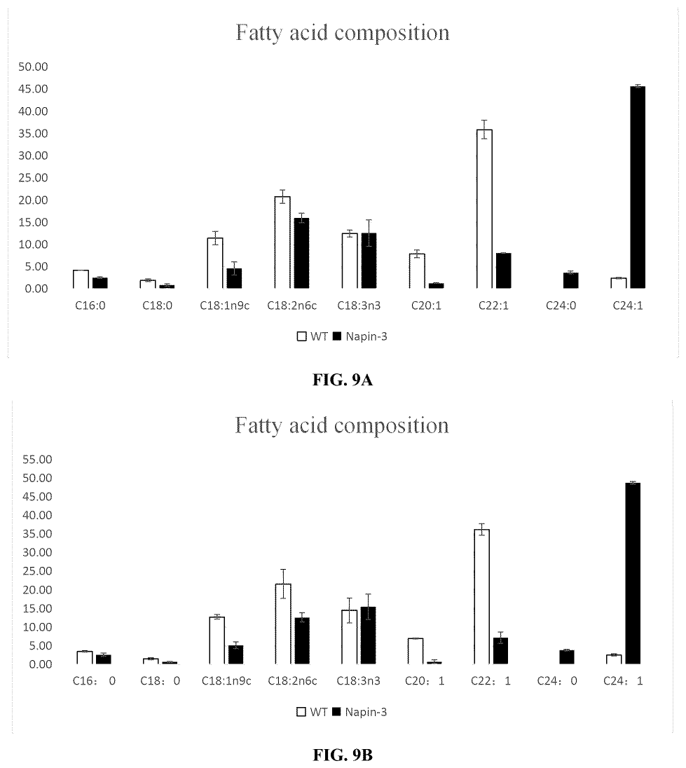

In the present disclosure, two lines with a NA ratio higher than 40% were also selected to analyze the changes in fatty acid composition, as shown in FIGS. 9A-B. The results showed that the scheme of the present disclosure could fully convert various substrates into NA, including ratios 18:1, 20:1, and 22:1, and the ratios 20:1 and 22:1 had the maximum conversion efficiency. 24:0 fatty acid, which the control itself did not contain, was also produced.

### 2. Determination of Fatty Acids in Napin-5 Transgenic Plants

The phenotypic traits of transgenic crops and recipient Brassica juncea sp. yellow seed were basically indistinguishable. The transferred genes were mainly to improve a nutritional quality of Brassica napus, and the nervonic acid content of transgenic lines was increased by overexpressing the CgKCS, SLC1-1, DGAT1, BnFAE1, and LdLPAAT genes. In order to determine the ratio of nervonic acid and other fatty acids, the composition of various fatty acids in the seeds (T1 generation) of 13 T0 positive individual plants and the seeds of Brassica juncea sp. yellow seed individual plants were determined by GC.

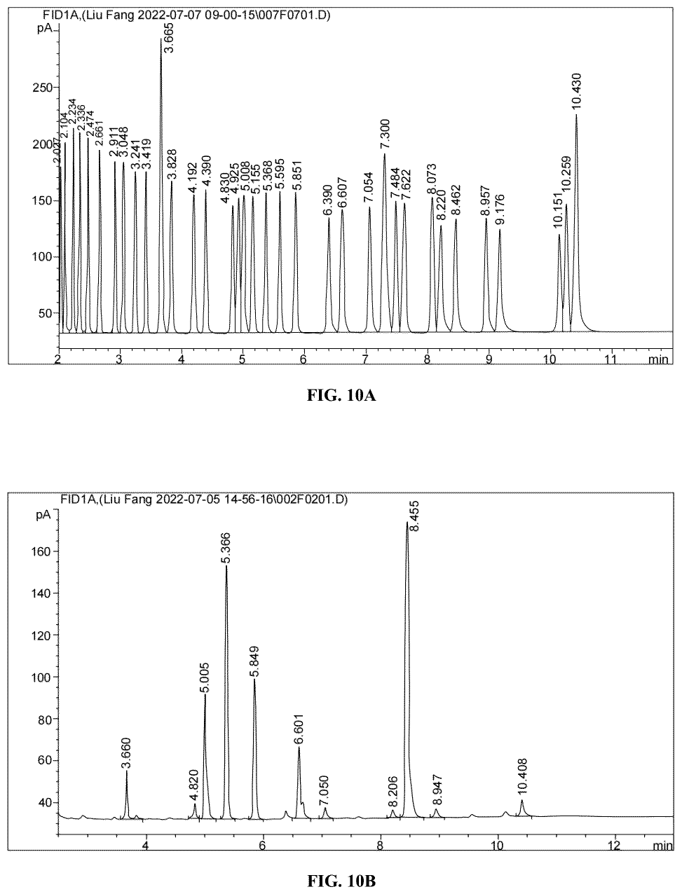

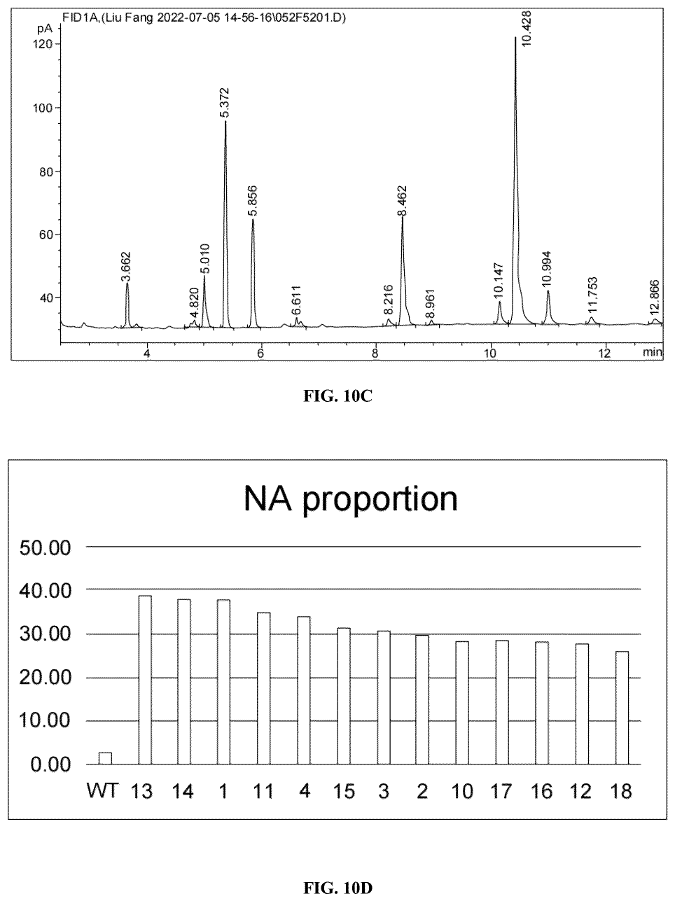

The inventors added an equal volume of 1 mg/ml nervonic acid methyl ester standard sample to the 25 mg/ml of 37 fatty acid methyl ester mixed standards for GC analysis. The additionally increased peak area indicated a peak position of nervonic acid at 10.430 min (FIG. 10A). The fatty acid composition of the control Brassica juncea sp. yellow seed was shown in FIG. 10B, where a peak position of nervonic acid was at 10.408 min. The fatty acid composition of the Napin-5-transgenic Brassica juncea sp. yellow seed control was shown in FIG. 10C, where a peak position of nervonic acid was at 10.428 min. GC results showed that the composition of nervonic acid and other fatty acids in the transgenic lines did change significantly (FIGS. 10A-10B). FIG. 10D showed that a series of T0 generation materials with high NA ratio were obtained. The seed oil in wild-type material had an NA ratio of 2.71%. Among the seed oil in T0 generation genetically engineered Brassica napus, there were 3 lines with NA ratio of 35% to 40%, 4 lines with NA ratio of 30% to 35%, and 6 lines with NA ratio of 25% to 30%. In these lines, NA accounted for 25.99% to 38.89% in fatty acids, and could reach 38.89% at the maximum level. The proportion of NA in fatty acids in T1 generation genetically engineered Brassica napus lines was up to 46.37%.

Calculated according to the same ratio of T0 generation, an estimated NA yield in soil seed could reach (163-183) g/kg (38.89%*42 to 47%*1000); calculated according to the same ratio of T1 generation, an estimated NA yield in soil seed could reach (195-218) g/kg (46.37%*42 to 47%*1000), which was higher than the highest level reported in plants so far, and might even achieve high production of NA considering the yield factor.

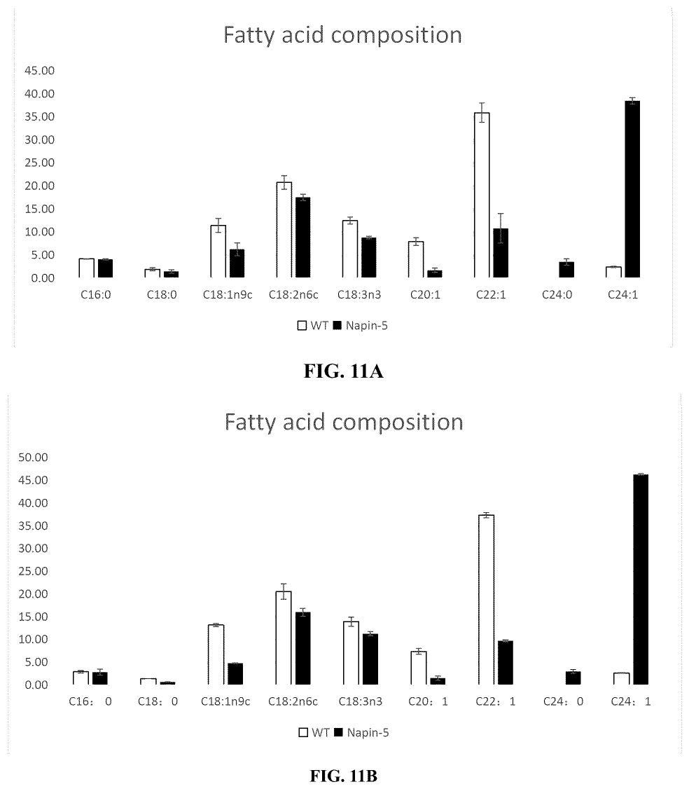

In the present disclosure, two lines with a NA ratio of about 38% were also selected to analyze the changes in fatty acid composition, as shown in FIGS. 11A-B. The results showed that the scheme of the present disclosure could fully convert various substrates into NA, including ratios 18:1, 20:1, and 22:1, and the ratios 20:1 and 22:1 had the maximum conversion efficiency. 24:0 fatty acid, which the control itself did not contain, was also produced.

Although the above example has described the present disclosure in detail, it is only a part of, not all of, the examples of the present disclosure. Other examples may also be obtained by persons based on the example without creative efforts, and all of these examples shall fall within the protection scope of the present disclosure.

What is claimed is:

1. A multigene co-expression plant vector, comprising an initial backbone of pBWA(V)BII and multiple exogenous gene expression cassettes; wherein
an exogenous gene in each of the exogenous gene expression cassettes is activated by a seed-specific expression promoter.

2. The plant vector according to claim 1, wherein the multiple exogenous gene expression cassettes are located in an independent T-DNA region of the plant vector, and the exogenous gene in each of the exogenous gene expression cassettes is a gene related to synthesis and assembly of nervonic acid and grease.

3. The plant vector according to claim 2, wherein the gene related to the synthesis and the assembly of the nervonic acid and the grease comprises a 3-ketoacyl-CoA synthase gene, a lysophosphatidic acid acyltransferase gene, and a diacylglycerol acyltransferase gene.

4. The plant vector according to claim 3, wherein the 3-ketoacyl-CoA synthase gene comprises BnFAE1 and/or CgKCS; the lysophosphatidic acid acyltransferase gene comprises SLC1-1 and/or LdLPAAT; and the diacylglycerol acyltransferase gene comprises DGAT1.

5. A three-gene co-expression plant vector for induced expression of nervonic acid and grease, wherein the three-gene co-expression plant vector is constructed based on an initial backbone of pBWA(V)BII and three exogenous gene expression cassettes located in a T-DNA region; and
the three exogenous gene expression cassettes comprise one 3-ketoacyl-CoA synthase gene expression cassette, one lysophosphatidic acid acyltransferase gene expression cassette, and one diacylglycerol acyltransferase gene expression cassette.

6. The three-gene co-expression plant vector according to claim 5, wherein the 3-ketoacyl-CoA synthase gene expression cassette comprises a Napin promoter, a CgKCS gene, and a Napin terminator;
the lysophosphatidic acid acyltransferase gene expression cassette comprises a Napin promoter, an SLC1-1 gene, and a Napin terminator; and
the diacylglycerol acyltransferase gene expression cassette comprises a Napin promoter, a DGAT1 gene, and a Napin terminator.

7. A five-gene co-expression plant vector for induced expression of nervonic acid and grease, wherein the five-gene co-expression plant vector is constructed based on an initial backbone of pBWA(V)BII and five exogenous gene expression cassettes located in a T-DNA region; and
the five exogenous gene expression cassettes comprise two 3-ketoacyl-CoA synthase gene expression cassettes, two lysophosphatidic acid acyltransferase gene expression cassettes, and one diacylglycerol acyltransferase gene expression cassette.

8. The five-gene co-expression plant vector according to claim 7, wherein 3-ketoacyl-CoA synthase genes in the two 3-ketoacyl-CoA synthase gene expression cassettes are a CgKCS gene and a BnFAE1 gene, respectively;
lysophosphatidic acid acyltransferase genes in the two lysophosphatidic acid acyltransferase gene expression cassettes are an SLC1-1 gene and a LdLPAAT gene, respectively; and
a diacylglycerol acyltransferase gene in the diacylglycerol acyltransferase gene expression cassette is a DGAT1 gene.
